(19) United States
(12) Patent Application Publication
(10) Pub. No.: US 2026/0355514 A1
(43) Pub. Date: Nov. 12, 2026

(54) CLOSED-LOOP LIMBIC-TARGETED SLOW-OSCILLATORY STIMULATION TO MODULATE COUPLED N3 AND REM SLEEP ARCHITECTURE

(71) Applicant: Brain Electrophysiology Laboratory Company, LLC, Eugene, OR (US)

(72) Inventors: Don M. Tucker, Eugene, OR (US); Phan Luu, Eugene, OR (US); Roman Shusterman, Lake Oswego, OR (US)

(21) Appl. No.: 19/669,925

(22) Filed: May 6, 2026

Related U.S. Application Data
(60) Provisional application No. 63/800,511, filed on May 6, 2025.

(51) Int. Cl. A61N 1/36; A61M 21/00; A61M 21/02; A61N 1/04

(52) U.S. Cl. CPC A61N 1/36025; A61M 21/02; A61N 1/0408; A61N 1/36031; A61N 1/36034; A61M 2021/0072; A61M 2205/50; A61M 2210/0693; A61M 2230/005; A61M 2230/10; A61M 2230/18

(57) ABSTRACT

A wearable closed-loop neuromodulation system improves coupled N3 and REM sleep architecture. The system acquires EEG, classifies sleep stage, detects a N2-to-N3 stimulation opportunity, and delivers low-amplitude slow-oscillatory transcranial electrical stimulation through a limbic-targeted electrode montage. A controller computes a N3-discharge index from N3 or slow-oscillation features. After a N3-discharge criterion or maximum stimulation dose is reached, the controller terminates, reduces, or withholds further N3-promoting stimulation and enters a REM-permissive interval. The controller monitors subsequent REM duration, REM continuity, REM latency, or other REM features and adapts stimulation timing or dose to improve a dual-stage sleep-architecture objective including both N3 and subsequent REM features while limiting arousal, wake, REM suppression, or N3 fragmentation. Optional impedance feedback computes skin-path-corrected intracranial impedance, FAST-ECS, or glymphatic-clearance indices to verify N2-to-N3 transition physiology and adjust stimulation policy.

### RELATED APPLICATION

This application claims the benefit of U.S. Provisional Patent Application No. 63/800,511, filed May 6, 2025, the entire disclosure of which is incorporated herein by reference.

### FIELD

The disclosure relates to non-invasive neuromonitoring and neuromodulation systems for improving human sleep architecture. More particularly, the disclosure relates to wearable, closed-loop systems that classify sleep stage from electroencephalography (EEG), detect a N2-to-N3 stimulation opportunity, deliver low-amplitude slow-oscillatory transcranial electrical stimulation (SO-tES) through a limbic-targeted montage, and control the stimulation policy to improve or preserve both stage N3 sleep and subsequent rapid eye movement (REM) sleep.

### BACKGROUND

Large slow oscillations (SOs), typically in a frequency band of about 0.4 Hz to about 1.2 Hz, are a defining physiological feature of non-rapid-eye-movement stage N3 sleep. SOs are associated with hippocampal-cortical replay, spindle coordination, memory consolidation, and sleep-dependent restorative processes. Prior efforts to stimulate or entrain sleep SOs generally treated N3 enhancement as the principal endpoint, and they did not provide a fully autonomous home-use controller that optimized whole-night N3 and REM organization as coupled sleep-architecture targets.

Some prior approaches assumed that large SOs of human deep sleep are generated primarily in frontal neocortex because frontal scalp electrodes often show large SO deflections. High-density EEG source-localization evidence and finite-element current modeling, however, support a model in which major large SOs arise from relatively focal limbic sources, including anterior parahippocampal and caudal orbitofrontal regions. Accordingly, stimulation montages that route current through frontopolar, inferolateral frontal, occipital, mastoid, nuchal, or suboccipital sites can be selected to couple current more effectively to ventral-limbic SO generators than conventional frontal-only or frontal-mastoid arrangements.

Known sleep architecture includes ultradian cycling between non-REM and REM sleep. Early-night sleep is dominated by NREM and N3, whereas later-night sleep contains more REM. Because NREM and REM are mutually exclusive instantaneous sleep states, it might be expected that enhancement of N3 would reduce REM, or that increasing one stage would occur at the expense of the other. Contrary to that expectation, the inventors observed that a short, closed-loop, N2-gated SO-tES intervention can increase N3 sleep and also increase subsequent REM sleep in the same night.

A need remains for a wearable system and controller that does not merely stimulate N3, but instead treats N3 and REM as coupled, sequential control targets. There is also a need for a controller that recognizes when N3 homeostatic or slow-oscillation processing has been sufficiently enhanced, terminates or reduces further N3-driving stimulation, and enters a REM-permissive mode so that subsequent REM duration, continuity, or probability is preserved or increased rather than sacrificed.

There is a further need for optional physiological feedback signals that can verify or adjust the sleep-architecture controller. Such signals can include slow-wave activity, SO density, SO-spindle coupling, arousal probability, cardiorespiratory features, skin-path-corrected intracranial impedance, a FAST-ECS measure of extracellular-space dynamics, and a glymphatic-clearance index. These signals can be used to detect N2-to-N3 transition windows, N3-discharge or NREM-load discharge, and subsequent REM-permissive intervals.

### SUMMARY

In one aspect, a wearable sleep-architecture modulation system includes an EEG front end, a transcranial electrical stimulation circuit, and one or more processors. The processors classify sleep stage from EEG, detect a N2-to-N3 stimulation opportunity, deliver slow-oscillatory stimulation through a limbic-targeted electrode montage, compute a N3-discharge index, and after the N3-discharge index or a maximum stimulation dose reaches a criterion, terminate, reduce, or withhold further N3-promoting stimulation and enter a REM-permissive interval.

In another aspect, the controller computes a dual-stage sleep-architecture objective comprising at least one N3 feature and at least one subsequent REM feature. The N3 feature can include N3 duration, slow-wave activity (SWA), SO density, SO amplitude, N3 continuity, SO-spindle coupling, or N2-to-N3 transition stability. The REM feature can include cumulative REM duration, REM duration in one or more cycles following stimulation, latency from N3 termination to REM onset, probability of REM following an enhanced N3 bout, REM continuity, REM fragmentation, phasic REM markers, or preservation of REM above a subject-specific floor.

In another aspect, the stimulation is delivered after detection of sustained N2 sleep, for example after at least about 1 minute to about 10 minutes of stable N2 sleep, preferably after about 4 minutes of stable N2 sleep. The stimulation can include alternating, biphasic, zero-DC, or substantially charge-balanced tES at about 0.1 Hz to about 2 Hz, preferably about 0.4 Hz to about 1.2 Hz, and in examples about 0.5 Hz or about 0.75 Hz. The stimulation amplitude can be sub-perceptual, for example not more than about 0.6 mA peak-to-peak per stimulation electrode, or other levels selected to avoid arousal, phosphenes, discomfort, or sleep fragmentation.

In another aspect, the electrode montage includes at least one anterior or frontal electrode and at least one nuchal, suboccipital, occipital, mastoid, neck, or posterior electrode. In preferred embodiments, a nuchal or suboccipital electrode is used to route current through high-conductivity cranial or craniocervical pathways, including the foramen magnum, toward limbic SO generators. A subject-specific or atlas-based head conductivity model can be used to select electrode positions, amplitude, polarity, waveform, or safety limits. A Foramen-Magnum Current-Occupancy Index (FM-COI) can optionally quantify current routing through a foramen-magnum region of interest.

In another aspect, the controller uses a state machine comprising a monitor state, a N2-to-N3 opportunity state, a NREM-drive or SO-synchronization state, a N3-discharge assessment state, a REM-permissive state, and an adaptation state. During the REM-permissive state, the controller withholds or reduces stimulation that would otherwise drive N3, monitors REM probability or REM features, and can adjust future stimulation timing or dose to increase a dual-stage N3-plus-REM objective while limiting arousal, wake after sleep onset, REM suppression, or N3 fragmentation.

In another aspect, the system learns subject-specific parameters over multiple nights. The controller can learn a user's usual N2-to-N3 latency, arousal threshold, amplitude tolerance, N3 response to stimulation, relationship between N3-discharge features and later REM features, REM floor, and responder type. The responder type can include a N3 responder, a REM responder, a dual-stage responder, an impedance responder, or a nonresponder. The controller can then personalize the N2 trigger, stimulation dose, maximum stimulation time, and REM-permissive interval.

In another aspect, optional impedance and glymphatic-clearance feedback are used. The system can acquire a single-frequency or multi-frequency impedance signal, compute a skin-path-corrected intracranial impedance estimate (SPC-Z), transform SPC-Z into a FAST-ECS or extracellular-space estimate, and compute a global or regional glymphatic-clearance index (GCI or RGCI). The controller can use these physiological measures as feedback to identify N2-to-N3 transition windows, verify an N3-discharge response, adjust stimulation, or assess whether subsequent REM is associated with decreased brain impedance or increased extracellular-space response.

The present disclosure therefore treats N3 and REM as opponent instantaneous states but complementary sequential control targets. Without being bound by theory, improving the timing and coherence of NREM SO activity can accelerate or deepen NREM homeostatic processing, after which a REM-permissive interval can allow subsequent REM reintegration to emerge with improved duration or continuity. The invention thereby provides a technical control architecture for the unexpected observation that N2-gated SO-tES can enhance N3 sleep and also enhance subsequent REM sleep.

### BRIEF DESCRIPTION OF THE DRAWINGS

FIG. 1. Positions of the tES electrodes (10, 20) optimized for applying electrical current to limbic areas of the brain, including current flow through the foramen magnum at the base of the skull.

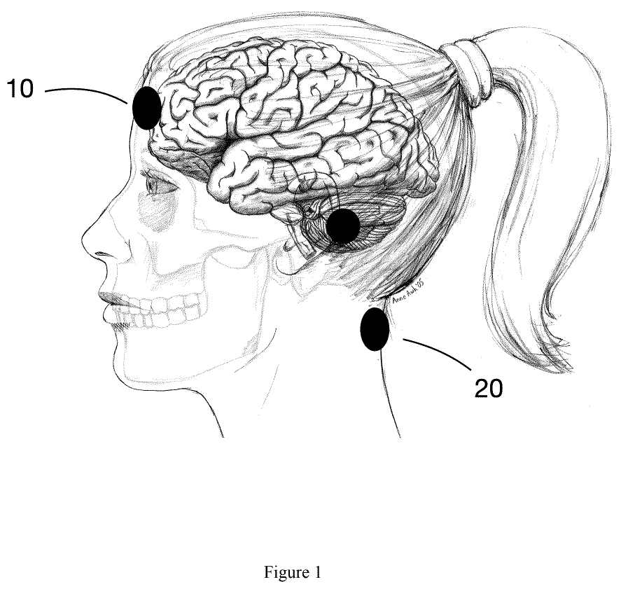

FIG. 2. System configuration including the headband tES electrodes (30), Convolutional Neural Net (CNN) sleep stage classifier (40), Closed-loop tES controller (50). The system allows continuous monitoring of the individual's sleep neurophysiology to apply optimized electrical currents in relation to specific sleep stages.

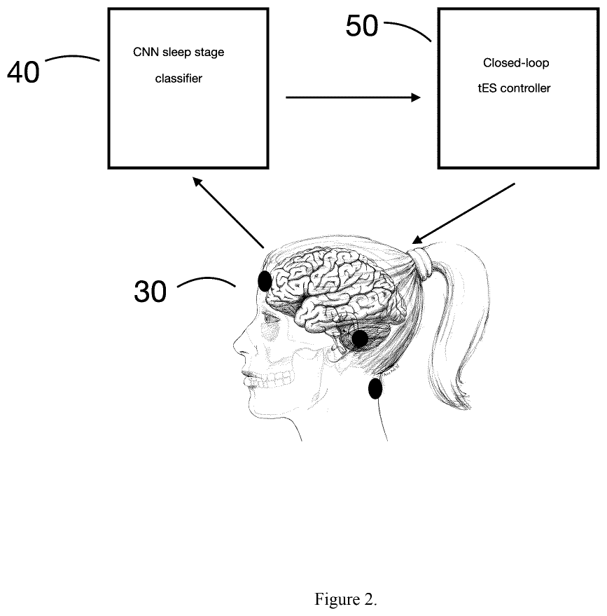

FIG. 3. Illustration of human sleep stage cycling, in which the NREM-REM cycles are weighted strongly toward NREM (slow wave sleep) early in the night, but progressively toward REM (REM sleep) later in the night. The critical factor is that effective stimulation of NREM sleep serves to enhance rapid transitions to REM, thereby combining the functional consolidation of memory that requires the coupling of NREM and REM effects on brain neurophysiology.

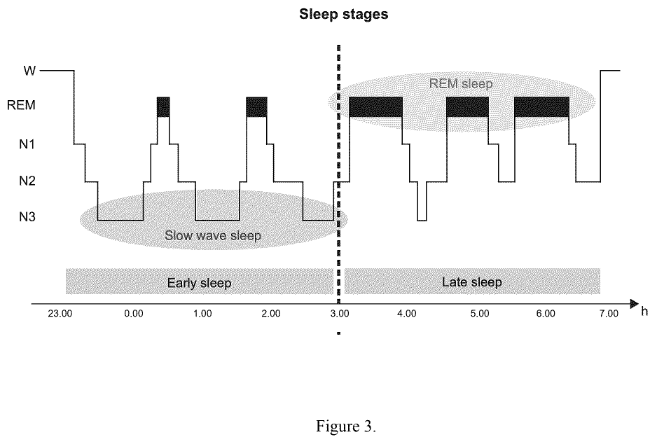

### DETAILED DESCRIPTION

### Definitions

As used herein, ‘N3’ refers to stage N3 non-REM sleep, also known as slow-wave sleep or deep sleep. ‘REM’ refers to rapid eye movement sleep. ‘N2’ refers to non-REM stage N2 sleep.

‘Slow-oscillatory transcranial electrical stimulation’ or ‘SO-tES’ refers to transcranial electrical stimulation having a fundamental frequency or rhythmic control component in a slow-oscillation band, for example about 0.1 Hz to about 2 Hz, about 0.4 Hz to about 1.2 Hz, about 0.5 Hz, or about 0.75 Hz. The waveform can be sinusoidal, square, ramped, pulsed, amplitude-modulated, phase-locked, burst-like, or another waveform selected to synchronize or enhance sleep SO activity.

‘Limbic-targeted’ means that a stimulation montage, waveform, or head model is selected to increase current density, electric field, or electric-field orientation in or near one or more limbic SO generators, including anterior parahippocampal cortex, caudal orbitofrontal cortex, anterior medial temporal cortex, hippocampal-adjacent tissue, ventral limbic cortex, or other cortical or subcortical targets associated with SO generation or regulation.

‘N2-to-N3 stimulation opportunity’ means a sleep interval during which a subject is in N2 sleep and the controller estimates that stimulation is likely to enhance N3 onset, N3 duration, N3 stability, SO density, or slow-wave activity without unacceptable arousal or sleep fragmentation. The opportunity can be determined from sleep stage, stage probability, time in N2, delta power, sigma activity, SO density, motion, heart rate, respiration, impedance, or other physiological features.

‘N3-discharge index’ or ‘NREM-load discharge index’ means a scalar, vector, classification, or state variable that estimates whether N3 homeostatic processing or NREM slow-oscillation processing has been sufficiently enhanced or stabilized. The index can include N3 minutes, SWA, SO amplitude, SO density, SO-spindle coupling, N3 continuity, transition stability, arousal probability, cardiorespiratory coupling, FAST-ECS response, SPC-Z decrease, GCI or RGCI increase, or a weighted combination thereof.

‘REM-permissive interval’ means a controller state or time interval after an N2-to-N3 stimulation opportunity, NREM-drive stimulation, or N3-discharge event in which further N3-promoting stimulation is withheld, reduced, changed, or gated to allow or preserve REM emergence. During the REM-permissive interval, the controller can monitor REM probability, REM onset, REM duration, REM continuity, phasic REM features, or REM fragmentation and can update future stimulation policy.

‘Dual-stage sleep-architecture objective’ means an objective function, score, controller reward, threshold rule, or optimization target comprising at least one N3 feature and at least one subsequent REM feature, optionally with penalty terms for wake, arousal, discomfort, motion, REM suppression, N3 fragmentation, total sleep time loss, or safety limits.

‘FAST-ECS’ means a fast-sampled or sleep-stage-stratified measure derived from intracranial impedance or conductivity to estimate extracellular-space dynamics. ‘SPC-Z’ means skin-path-corrected intracranial impedance. ‘GCI’ and ‘RGCI’ mean global and regional glymphatic-clearance indices derived from impedance, sleep stage, regional sensitivity maps, or other physiological features.

### System Overview

A wearable device can be provided as a headband, cap, strap, patch, neckband, or other head-worn assembly configured for unattended home or bedside use. The device can include EEG electrodes, stimulation electrodes, electrode drivers, an EEG front end, optional impedance-measurement circuits, an inertial sensor, a respiration or cardiac sensor, a battery, wireless communication, and one or more processors. The processors can execute sleep-stage classification, artifact rejection, stimulation control, safety monitoring, data logging, and adaptive personalization.

The EEG module can include at least two EEG channels, preferably frontopolar and mastoid or frontal and mastoid derivations. Additional channels can include Fp1, Fp2, M1, M2, a common forehead electrode, occipital or neck electrodes, electrooculographic derivations, or electromyographic derivations. A classifier can classify sleep stage in 30-second epochs or in shorter overlapping windows, and can output probabilities for wake, N1, N2, N3, and REM.

The stimulation module can include one or more constant-current or voltage-limited drivers. The drivers can deliver charge-balanced, DC-bias-free, ramped SO-tES while recording EEG during off intervals or using stimulation-artifact removal during stimulation. Stimulation can be applied in one or more blocks, for example five blocks of five minutes, five blocks of four minutes, one or more blocks of one to ten minutes, or a continuously adjusted waveform. Off intervals can be zero to several minutes and can be used for EEG or impedance assessment.

The processor can operate locally in the wearable device, in a bedside hub, in a mobile device, or in a cloud-connected system. The processor can execute a real-time controller and can transmit summary data to a clinician, investigator, or caregiver. However, no human observer is required to identify the N2-to-N3 opportunity or to initiate stimulation.

### Electrode Montage and Current Routing

The stimulation montage can include at least one anterior electrode located on the forehead, frontopolar scalp, inferolateral frontal scalp, near Fp1, Fp2, F9, F10, or other anterior site, and at least one posterior electrode located at an occipital, mastoid, nuchal, suboccipital, cervical, or upper-neck site. In a preferred embodiment, a nuchal or suboccipital electrode is used to route current through craniocervical pathways, including the foramen magnum, toward limbic SO generators.

Because a biphasic or alternating waveform can reverse source and sink polarity during a cycle, references herein to ‘source’ and ‘sink’ electrodes are illustrative. The controller can use either polarity, alternating polarity, monopolar, bipolar, multipolar, or high-definition configurations. In some embodiments, anterior electrodes act as sources during one phase and nuchal electrodes act as returns, and the polarity reverses during the opposite phase.

A head conductivity model can be used to select montage parameters. The model can be subject-specific, MRI-based, CT-based, atlas-based, anthropometric, impedance-calibrated, or a hybrid. The model can estimate electric field, current density, field orientation, foramen-magnum current occupancy, cranial-nerve exposure, hindbrain field, skin current density, optic-canal routing, and current density in limbic targets. The model can be computed before use, updated during use, or approximated by a learned mapping from head measurements and electrode impedances.

In some embodiments, a Foramen-Magnum Current-Occupancy Index (FM-COI) is computed as a ratio or fraction of current, current density, or electric-field energy crossing or occupying a foramen-magnum region of interest relative to total injected current or a reference volume. Stimulation can be limited to montages meeting a minimum FM-COI or adjusted to increase FM-COI while respecting safety caps.

### Sleep-Stage Classification and N2-to-N3 Opportunity Detection

The controller can classify sleep stage using a convolutional neural network, recurrent neural network, transformer, hidden Markov model, random forest, support vector machine, rule-based classifier, or hybrid classifier. In a preferred embodiment, a CNN-based classifier processes EEG windows and adjacent context to output sleep-stage probabilities. The classifier can be trained on polysomnography data from multiple laboratories, devices, age ranges, or patient groups.

The controller can detect sustained N2 when N2 probability exceeds a threshold for at least a defined duration, such as at least about 60 seconds, 120 seconds, 180 seconds, 240 seconds, 300 seconds, or 600 seconds. In one preferred embodiment, the controller initiates stimulation after about 4 consecutive minutes of N2 sleep. The N2-to-N3 opportunity can also require low motion, acceptable electrode impedance, low arousal probability, stable respiration, absence of REM, absence of wake, and absence of recent stimulation or recent arousal.

In some embodiments, the controller estimates a probability of N3 onset within a future window, for example within 5, 10, 15, 20, or 30 minutes. The probability can be determined from time in N2, delta power, sigma power, SO-like K-complexes, autonomic features, prior-night history, circadian time, cycle number, and subject-specific transition statistics. Stimulation can be initiated only if the probability exceeds a threshold and sleep-architecture safety constraints are satisfied.

### Stimulation Protocols

SO-tES can be delivered at a frequency selected to synchronize SOs, for example about 0.4 Hz to about 1.2 Hz. In examples, the stimulation frequency is about 0.5 Hz or about 0.75 Hz. The stimulation amplitude can be about 0.05 mA to about 1.0 mA, about 0.1 mA to about 0.8 mA, or not more than about 0.6 mA peak-to-peak per stimulation electrode. The amplitude can be adjusted based on subject response, electrode impedance, arousal risk, sleep stage, or head-model limits.

The stimulation can be delivered in multiple blocks. One example uses five blocks of five minutes separated by one-minute off intervals. Another example uses five blocks of four minutes with quiescent intervals not exceeding the immediately preceding block. Other protocols can use total on-time of about 5 minutes to about 40 minutes, preferably about 15 minutes to about 30 minutes, with ramp-up and ramp-down periods to reduce arousal.

Stimulation can be terminated or reduced when a maximum dose is reached, when an arousal or wake probability exceeds a threshold, when REM probability exceeds a threshold, when a N3-discharge index reaches a threshold, when N3 fragmentation is detected, when discomfort or electrode impedance is unacceptable, or when electrical safety limits are approached.

### Closed-Loop N3-to-REM Controller

A preferred controller is a state machine. In a monitor state, the system continuously or periodically classifies sleep stage and checks data quality. In an opportunity state, the controller identifies sustained N2 or another N2-to-N3 opportunity. In a NREM-drive state, the controller delivers SO-tES to enhance or synchronize N3 slow oscillations. In a N3-discharge assessment state, the controller computes a N3-discharge index and determines whether sufficient NREM processing has occurred. In a REM-permissive state, the controller withholds, reduces, or modifies further N3-driving stimulation so that REM can emerge or be preserved. In an adaptation state, the controller updates individualized parameters from the night's response and prior nights.

The REM-permissive state is not merely an absence of stimulation. It is an active controller policy. The controller can monitor REM probability, REM latency, REM continuity, phasic REM features, respiration, heart rate, body movement, EEG beta activity, and arousal probability. If the controller detects that the subject is likely to enter REM, stimulation that would promote N3 can be suppressed. If REM duration falls below a subject-specific floor, future nights can use a shorter NREM-drive dose, an earlier termination criterion, a longer REM-permissive interval, or a modified stimulation frequency or amplitude.

The REM-permissive interval can have a fixed duration, such as about 5 minutes to about 180 minutes, or can be event-based, for example continuing until REM onset, until REM termination, until the next N2-to-N3 opportunity, until a predicted REM window has passed, or until the dual-stage objective indicates that further N3 stimulation would be beneficial without suppressing REM.

The N3-discharge index can be computed in many ways. One example is a weighted sum of normalized features: N3 duration, SWA increase, SO density increase, SO amplitude, SO-spindle coupling, reduced arousal probability, N3 continuity, and optional FAST-ECS or GCI response. Another example is a classifier trained to predict whether additional N3-driving stimulation is likely to improve the dual-stage objective or instead increase arousal, N3 fragmentation, or REM suppression.

A controller can treat NREM and REM as complementary sequential targets rather than competing stage minutes. NREM-drive stimulation is used when N2-to-N3 physiology indicates an opportunity to enhance N3, but the controller then releases or permits REM by terminating, reducing, or withholding further N3-driving stimulation after N3 processing has been stabilized or after a maximum dose. This N3-to-REM sequencing provides a technical explanation and implementation of the observed dual-stage benefit.

### Dual-Stage Sleep-Architecture Objective

A dual-stage objective can be optimized in real time, within-night, or across nights. In one non-limiting embodiment, the objective D is: D=w1*N3_minutes+w2*SWA+w3*SO_coupling+w4*Subsequent_REM_minutes+w5*REM_continuity−w6*Wake_after_sleep_onset−w7*Arousals−w8*N3_fragmentation−w9*REM_suppression. The weights can be fixed, subject-specific, clinician-selected, learned from prior nights, or adapted to a clinical objective.

The N3 feature and REM feature need not be measured in the same sleep cycle. In some embodiments, the REM feature is measured in a REM period following the stimulated N3 bout. In other embodiments, the REM feature is cumulative REM for the remainder of the night, REM in the next one or more ultradian cycles, REM continuity in later sleep, or preservation of a subject-specific REM floor. The controller can use predicted REM features before they occur and observed REM features after they occur.

Penalty terms can enforce sleep-architecture safety. For example, the controller can avoid protocols that increase N3 but reduce REM below a subject-specific floor, increase wake after sleep onset, increase arousal index, cause N3 fragmentation, cause excessive total sleep time extension or curtailment, produce discomfort, or violate electrical safety caps.

### Adaptive Personalization

The system can personalize stimulation over nights. The controller can estimate the subject's baseline N2-to-N3 latency, baseline N3 duration, baseline REM duration, N3 response to stimulation, REM response to stimulation, dose-response curve, arousal threshold, electrode impedance pattern, preferred montage, and FAST-ECS or GCI response. The controller can use these features to classify the subject as a N3 responder, REM responder, dual-stage responder, impedance responder, or nonresponder.

A learning algorithm can update N2 duration thresholds, stimulation onset timing, frequency, amplitude, block duration, maximum dose, N3-discharge threshold, and REM-permissive interval. For example, if a subject shows increased N3 but reduced subsequent REM, the controller can shorten the stimulation dose or increase the REM-permissive interval. If a subject shows inadequate N3 response but preserved REM, the controller can modestly increase stimulation dose within safety limits. If a subject shows arousals, the controller can reduce amplitude, add longer ramps, change frequency, or restrict stimulation to earlier sleep cycles.

The controller can operate differently by sleep cycle. Early-night cycles can have a higher N3 drive target, while later-night cycles can have a higher REM preservation or REM enhancement target. The controller can also account for age, sleep disorder, medication, circadian phase, prior-night sleep debt, cognitive status, or clinical diagnosis.

### Optional FAST-ECS, SPC-Z, GCI, and RGCI Feedback

In some embodiments, the wearable includes an impedance front end that injects a low-amplitude alternating current and measures impedance at a single frequency, such as about 7 Hz to about 8 Hz, or at multiple frequencies. The impedance circuit can be separate from the stimulation circuit or time-multiplexed with stimulation. The impedance signal can be synchronized with sleep stage, stimulation timing, respiration, heart rate, and arousal features.

Skin-path correction can be used to estimate intracranial impedance. In one example, a brain-inclusive path such as Fp2-to-M1 is corrected by subtracting one-half the sum of nearby skin-dominated paths such as Fp2-to-Fp1 and M1-to-M2. More generally, a corrected impedance estimate can be computed as Z_brain_hat=Z_meas−alpha*Z_skin1−beta*Z_skin2−gamma*Z_skin3, where the coefficients are fixed, learned, stage-conditioned, subject-specific, or estimated by adaptive filtering or regression.

The controller can interpret decreased skin-path-corrected intracranial impedance as increased extracellular conductive volume or increased extracellular-space response. A FAST-ECS feature can be computed as a sign-adjusted, normalized, stage-conditioned, regional, or time-resolved feature derived from corrected impedance. A GCI or RGCI can be computed from corrected impedance, regional sensitivity maps, head-model priors, sleep stage, and physiological driver features.

FAST-ECS, SPC-Z, GCI, or RGCI feedback can be used to identify N2-to-N3 transition windows, verify that SO-tES produced an expected N3 or transition-associated physiological response, determine a N3-discharge threshold, detect REM-associated physiological response, and adjust future stimulation. These impedance and glymphatic features are optional. The dual-stage sleep-architecture controller can operate using EEG and sleep-stage features alone, with impedance added as a physiological verification or biomarker layer.

### Safety and Quality Control

Electrical safety limits can include current, voltage, charge density, skin current density, hindbrain electric field, cranial nerve electric field, duty cycle, total dose, impedance, electrode contact quality, and battery limits. Sleep-architecture safety limits can include REM preservation above a floor, maximum arousal index, maximum wake after sleep onset, maximum N3 fragmentation, avoidance of stimulation during wake, and avoidance of stimulation during REM unless a REM-specific stimulation mode is intentionally selected.

The controller can reject epochs having excessive motion, poor electrode contact, low signal-to-noise ratio, inconsistent impedance phase, suspected electrodermal artifact, excessive muscle artifact, stimulation artifact, or classification uncertainty. If quality is inadequate, the controller can suspend stimulation and resume monitoring until a valid opportunity is detected.

### Non-Limiting Mechanistic Rationale

Without being bound by theory, the inventors believe that timed SO-tES during a N2-to-N3 opportunity promotes NREM slow-oscillation synchronization and associated inhibitory differentiation of recent experience. This can accelerate, deepen, or stabilize NREM homeostatic processing. After sufficient NREM processing, continued N3-driving stimulation can be unnecessary or counterproductive for whole-night architecture. The controller therefore enters a REM-permissive state, allowing subsequent REM processes of reintegration and generative consolidation to emerge with improved duration or continuity.

This rationale treats NREM and REM as opponent in instantaneous state expression but complementary in whole-night computation. Better NREM partitioning and slow-oscillation organization can improve the eligibility landscape from which REM can recruit later integrative processing. Thus, a short N2-gated NREM intervention can legitimately increase later REM rather than suppressing it.

### Experimental Examples

Example 1—At-home WISP stimulation and dual-stage sleep architecture. Healthy adult participants used a head-worn WISP system at home. Sleep stage was scored using an automated classifier. Active stimulation was delivered during a N2-to-N3 opportunity, and sham nights were used for comparison. In an updated analysis excluding one outlier, stimulation produced significantly longer REM sleep than sham, with REM increasing from about 32.81 minutes to about 49.75 minutes, and significantly longer N3 sleep than sham, with N3 increasing from about 69.84 minutes to about 84.94 minutes. Total sleep time also increased, while N1, N2, and wake after sleep onset were not significantly different. These results show a dual-stage benefit rather than a simple substitution of N3 for REM.

Example 2—Transition-associated impedance response. In a sleep impedance analysis using skin-path-corrected intracranial impedance, stimulation nights showed lower estimated brain impedance than sham nights in several sleep-stage comparisons, including N2. When aligned to first N3 onset, the stimulation-minus-sham difference was largest about 15 minutes after N3 onset and was consistent with a transition-biased effect at the N2-to-N3 transition. This supports using transition physiology, N3-discharge indices, and optional FAST-ECS feedback as controller features.

Example 3—Controller operation. A subject applies a wearable headband before sleep. The controller monitors EEG and identifies stable N2 for about 4 minutes. The controller delivers SO-tES at about 0.75 Hz through anterior and nuchal electrodes in several blocks. After the maximum dose or a N3-discharge threshold is reached, the controller enters a REM-permissive state and withholds further N3-driving stimulation. The controller logs subsequent REM duration and continuity, updates the dual-stage objective, and adjusts the next night's stimulation policy.

Example 4—Subject-specific REM preservation. If a subject's first active night shows increased N3 but REM duration below the subject's baseline REM floor, the controller can reduce total on-time, increase the N3-discharge threshold sensitivity, or lengthen the REM-permissive interval on later nights. If REM increases but N3 does not, the controller can adjust stimulation onset earlier within stable N2, increase amplitude within safety caps, or modify the stimulation frequency.

### Alternative Embodiments

Although many examples describe N2-gated stimulation, stimulation can be initiated during stable N2, at the N2-to-N3 transition, during early N3, during a predicted N3 opportunity, or during a stage-conditioned glymphatic-clearance window. Although many examples describe withholding stimulation to permit REM, the REM-permissive interval can alternatively use a different waveform, lower amplitude, different target, respiratory phase-locking, or a sensory cue intended to preserve rather than suppress REM.

Although many examples describe healthy adult users, the system can be used in older adults, persons with mild cognitive impairment, persons at risk for neurodegenerative disease, persons with post-traumatic stress symptoms, persons with insufficient N3 or REM, military or shift-work populations, or other users for whom improvement of N3 and REM sleep architecture is desired. Clinical indications and claims can be adjusted by counsel according to regulatory and prosecution strategy.

The embodiments described herein are examples. Any feature described in connection with one embodiment can be used with another embodiment unless the context indicates otherwise. The scope of the invention is defined by the claims, not by any particular scientific theory or experimental example.

What is claimed is:

1. A wearable sleep-architecture modulation system comprising: an EEG front end configured to acquire EEG from a sleeping subject; a transcranial electrical stimulation circuit coupled to at least one anterior electrode and at least one posterior, nuchal, suboccipital, occipital, mastoid, or neck electrode; and one or more processors configured to: classify sleep stage from the EEG; detect a N2-to-N3 stimulation opportunity; deliver slow-oscillatory transcranial electrical stimulation through a limbic-targeted electrode montage during the N2-to-N3 stimulation opportunity; compute a N3-discharge index from one or more N3 or slow-oscillation features; after the N3-discharge index reaches a criterion or after a maximum stimulation dose is reached, terminate, reduce, or withhold further N3-promoting stimulation and enter a REM-permissive interval; monitor at least one subsequent REM feature during the same night; and update or select a stimulation policy according to a dual-stage sleep-architecture objective comprising at least one N3 feature and the at least one subsequent REM feature.

2. The system of claim 1, wherein the N2-to-N3 stimulation opportunity comprises stable N2 sleep for at least about 1 minute to about 10 minutes.

3. The system of claim 2, wherein the N2-to-N3 stimulation opportunity comprises at least about 4 consecutive minutes of N2 sleep.

4. The system of claim 1, wherein the sleep-stage classifier comprises a convolutional neural network configured to classify epochs or overlapping windows as wake, N1, N2, N3, or REM.

5. The system of claim 1, wherein the slow-oscillatory transcranial electrical stimulation has a fundamental frequency between about 0.4 Hz and about 1.2 Hz.

6. The system of claim 5, wherein the fundamental frequency is about 0.5 Hz or about 0.75 Hz.

7. The system of claim 1, wherein the stimulation is sub-perceptual and delivers not more than about 0.6 mA peak-to-peak per stimulation electrode.

8. The system of claim 1, wherein the stimulation is delivered in a plurality of blocks separated by one or more off intervals used for EEG, impedance, artifact, or arousal assessment.

9. The system of claim 8, wherein the plurality of blocks comprises five stimulation blocks of about four minutes or about five minutes each.

10. The system of claim 1, wherein the limbic-targeted electrode montage is selected to increase current density or electric field in anterior parahippocampal cortex, caudal orbitofrontal cortex, anterior medial temporal cortex, hippocampal-adjacent tissue, or ventral limbic cortex.

11. The system of claim 1, wherein the at least one posterior, nuchal, suboccipital, occipital, mastoid, or neck electrode comprises a nuchal or suboccipital electrode configured to route current through or adjacent to the foramen magnum.

12. The system of claim 1, wherein the one or more processors are configured to obtain a subject-specific or atlas-based head conductivity model and use the model to select electrode position, stimulation amplitude, polarity, waveform, or safety limits.

13. The system of claim 12, wherein the one or more processors compute a Foramen-Magnum Current-Occupancy Index and adjust the stimulation policy based at least in part on the Foramen-Magnum Current-Occupancy Index.

14. The system of claim 1, wherein the N3-discharge index comprises one or more of N3 duration, slow-wave activity, slow-oscillation amplitude, slow-oscillation density, N3 continuity, N2-to-N3 transition stability, slow-oscillation-spindle coupling, arousal probability, respiratory coupling, cardiac coupling, skin-path-corrected intracranial impedance, FAST-ECS response, GCI, or RGCI.

15. The system of claim 1, wherein the REM-permissive interval comprises a time interval during which the one or more processors suppress additional N3-promoting stimulation when REM probability exceeds a threshold.

16. The system of claim 1, wherein the at least one subsequent REM feature comprises REM duration in one or more sleep cycles following the stimulation, cumulative REM duration after the stimulation, REM latency after an enhanced N3 bout, REM continuity, REM fragmentation, probability of REM following N3, or a phasic REM marker.

17. The system of claim 1, wherein the dual-stage sleep-architecture objective includes a REM floor and a penalty term for suppressing REM below the REM floor.

18. The system of claim 1, wherein the dual-stage sleep-architecture objective includes one or more penalty terms for wake after sleep onset, arousals, N3 fragmentation, motion, discomfort, poor electrode contact, or electrical safety-limit approach.

19. The system of claim 1, wherein the one or more processors are configured to learn subject-specific parameters over multiple nights, the parameters comprising N2-to-N3 latency, arousal threshold, N3response, REM response, stimulation dose response, REM floor, or a dual-stage responder phenotype.

20. The system of claim 19, wherein the dual-stage responder phenotype comprises a N3 responder, a REM responder, a dual-stage responder, an impedance responder, or a nonresponder.

21. The system of claim 1, further comprising an impedance front end configured to acquire a single-frequency or multi-frequency impedance signal during sleep.

22. The system of claim 21, wherein the one or more processors compute skin-path-corrected intracranial impedance by suppressing one or more skin-dominated impedance paths from a brain-inclusive impedance path.

23. The system of claim 22, wherein the skin-path-corrected intracranial impedance is computed according to Z_brain_hat=Z_meas−alpha*Z_skin1−beta*Z_skin2-gamma*Z_skin3, wherein alpha, beta, and gamma are fixed, learned, stage-conditioned, or subject-specific coefficients.

24. The system of claim 21, wherein the one or more processors compute a FAST-ECS feature, global glymphatic-clearance index, or regional glymphatic-clearance index and use the feature or index to adjust stimulation timing, stimulation dose, the N3-discharge criterion, or the REM-permissive interval.

25. The system of claim 1, wherein the one or more processors are configured to reject or suspend stimulation during epochs having excessive motion, poor electrode impedance, low signal-to-noise ratio, suspected electrodermal artifact, wake probability above a threshold, or arousal probability above a threshold.

26. A method of modulating coupled N3 and REM sleep architecture in a sleeping subject, the method comprising: acquiring EEG from the subject using a wearable device; classifying sleep stage from the EEG in real time; detecting a N2-to-N3 stimulation opportunity; delivering slow-oscillatory transcranial electrical stimulation through a limbic-targeted electrode montage during the N2-to-N3 stimulation opportunity; computing a N3-discharge index from one or more N3 or slow-oscillation features; responsive to the N3-discharge index or a maximum stimulation dose, terminating, reducing, or withholding further N3-promoting stimulation; entering a REM-permissive interval; monitoring at least one subsequent REM feature; and adapting a stimulation policy to improve a dual-stage objective comprising at least one N3 feature and at least one subsequent REM feature.

27. The method of claim 26, wherein detecting the N2-to-N3 stimulation opportunity comprises detecting at least about 4 consecutive minutes of N2 sleep.

28. The method of claim 26, wherein delivering the stimulation comprises delivering stimulation at about 0.4 Hz to about 1.2 Hz and not more than about 0.6 mA peak-to-peak per stimulation electrode.

29. The method of claim 26, wherein delivering the stimulation comprises delivering five blocks of stimulation separated by one-minute off intervals.

30. The method of claim 26, further comprising selecting the limbic-targeted electrode montage from a head conductivity model to increase current density in anterior parahippocampal or caudal orbitofrontal cortex.

31. The method of claim 26, wherein entering the REM-permissive interval comprises withholding slow-oscillatory stimulation until REM onset, until REM termination, until a predicted REM window passes, or until a subsequent N2-to-N3 stimulation opportunity is detected.

32. The method of claim 26, further comprising preserving REM minutes above a subject-specific REM floor while increasing or preserving N3 duration, slow-wave activity, or slow-oscillation-spindle coupling.

33. The method of claim 26, further comprising acquiring an impedance signal during sleep, computing skin-path-corrected intracranial impedance, and using a decrease in the skin-path-corrected intracranial impedance during N2-to-N3 transition, N3, or REM as feedback for stimulation-policy adaptation.

34. The method of claim 26, further comprising classifying the subject as a N3 responder, REM responder, dual-stage responder, impedance responder, or nonresponder based on one or more prior nights.

35. A non-transitory computer-readable medium storing instructions that, when executed by one or more processors of a wearable sleep-architecture modulation system, cause the one or more processors to:
classify sleep stage from EEG; detect a N2-to-N3 stimulation opportunity; control delivery of slow-oscillatory transcranial electrical stimulation through a limbic-targeted electrode montage; compute a N3-discharge index; terminate, reduce, or withhold further N3-promoting stimulation after the N3-discharge index or a maximum stimulation dose reaches a criterion; enter a REM-permissive interval;
monitor a subsequent REM feature; and adapt a stimulation policy according to a dual-stage sleep-architecture objective comprising at least one N3 feature and at least one subsequent REM feature.

36. The non-transitory computer-readable medium of claim 35, wherein the instructions further cause the one or more processors to learn subject-specific stimulation timing, dose, N3-discharge criteria, and REM-permissive interval parameters over a plurality of nights.

37. The non-transitory computer-readable medium of claim 35, wherein the instructions further cause the one or more processors to compute skin-path-corrected intracranial impedance or FAST-ECS and use the impedance or FAST-ECS to adjust the stimulation policy.
